(19) United States
(12) Patent Application Publication
(10) Pub. No.: US 2014/0242149 A1
(43) Pub. Date: Aug. 28, 2014

(54) High-Viscosity Silicone Adhesive

(71) Applicant: Dow Corning Corporation, Midland, MI (US)

(72) Inventors: David Gantner, Midland, MI (US); Roger A. Gibas, Bay City, MI (US); Do-Lung Pan, Taoyuan Hsien (TW); Xavier Thomas, Famars (FR); Christine A. Weber, Pinconning, MI (US)

(73) Assignee: Dow Corning Corporation, Midland, MI (US)

(21) Appl. No.: 14/351,837

(22) PCT Filed: Oct. 12, 2012
(86) PCT No.: PCT/US2012/059951

§ 371 Date: Apr. 14, 2014

Related U.S. Application Data
(60) Provisional application No. 61/546,358, filed on Oct. 12, 2011.

(51) Int. Cl. A61L 15/26; A61L 15/18; A61L 15/58

(52) U.S. Cl. CPC A61L 15/26; A61L 15/58; A61L 15/18
USPC 424/445; 514/769; 523/105; 427/256; 427/2.31

(57) ABSTRACT

Provided in various embodiments are high viscosity, shear-thinning silicone compositions that can be pattern coated directly onto a substrate and silicone compositions having high-density particles suspended in an adhesive gel. The silicone compositions contain a thixotropic additive, such as a hydrogenated vegetable oil. The silicone compositions may be applied on a substrate for use in medical devices or wound dressings.

### FIELD OF THE INVENTION

The invention relates to high viscosity, shear-thinning silicone compositions that can be pattern coated directly onto a substrate, adhesive gel compositions having high-density particles suspended in an adhesive gel, and the use of such gels in medical dressings and applications where a suitable skin-facing adhesive material is desired.

### BACKGROUND OF THE INVENTION

Most advanced wound care applications demand that exudate be removed from the patient's skin in order to prevent irritation and facilitate healing. While silicone gel adhesives are often used to provide some level of occlusiveness, locking in too much moisture over time can lead to wound maceration. The moisture level can be managed, to some degree, by making the silicone layer discontinuous. Several types of silicone dressings that have a discontinuous silicone layer have gained increasing acceptance in treating wounds such as pressure sores and ulcers. Conventional wound care products incorporate the use of polymeric foams, polymeric films, particulate and fibrous polymers, and/or non-woven and woven fabrics. Dressings with the right combination of these components promote wound healing by providing a moist environment, while removing excess exudate and toxic components, and further serve as a barrier to protect the wound from secondary bacterial infection.

However, these dressings often involve several layers of films and liners and complex preparation steps in order to produce a product that is capable of achieving the desired level of discontinuity while also retaining the desired level of adhesiveness in the silicone dressing. A typical silicone wound dressing construction starts with a multi-layer rollstock that contains a release liner, a silicone adhesive gel, an optional primer, a polyurethane film, and a paper liner. The paper liner is removed, and the silicone rollstock is then laminated on the absorbent media (such as a foam substrate), and topped with a suitable backing material. Additionally, many manufacturing processes employ further steps of perforating the carrier film to introduce holes into the film, further adding to the cost.

Therefore, what is needed in the art is a silicone coated wound dressing that can be prepared by a simpler, less expensive process that involves fewer materials while achieving the same or similar advantages of conventional silicone dressings. This invention answers that need.

### SUMMARY OF THE INVENTION

This invention relates to silicone compositions that are flowable in the presence of an applied stress and can be pattern coated directly onto a substrate. The silicone compositions exhibit high viscosity and shear-thinning properties.

The silicone composition may be prepared by mixing (a) at least one organopolysiloxane, (b) at least one SiH-containing organopolysiloxane, (c) a thixotropic additive, and (d) a hydrosilyation catalyst. The thixotropic additive may be present in amounts ranging from about 1 to about 15 wt. % based on the total wt. % of the silicone composition. The silicone composition is cured to form a silicone adhesive gel. The silicone composition exhibits (i) viscosity ranging from about 7000 cP to about 5,000,000 cP and (ii) shear thinning behavior, as determined by the rheological profile. Once the silicone composition is pattern coated onto a substrate, the pattern of the coating is able to be maintained upon application. The silicone adhesive gel exhibits (i) adhesiveness ranging from about 0.2 N to about 4 N and (ii) cohesive strength, as determined by the peel adhesion test.

The invention also relates to a silicone composition having high-density particles suspended in an adhesive gel. The silicone composition comprises (a) at least one organopolysiloxane, (b) at least one SiH-containing organopolysiloxane, and (c) about 0.1 to about 3 wt. % of thixotropic additive.

The thixotropic additive has the formula (Formula I):

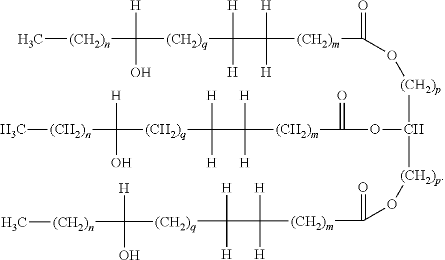

Variables m, n, p, and q are each, independently, an integer ranging from 1-10. The silicone composition is capable of suspending high-density particles. The invention also relates to a method of introducing into the silicone composition a thixotropic additive having the above formula.

Additional aspects of the invention will be apparent to those of ordinary skill in the art in view of the detailed description of various embodiments, a brief description of which is provided below.

### DETAILED DESCRIPTION

This invention relates to a high viscosity, shear-thinning silicone composition that can be pattern coated directly onto a substrate. The high viscosity silicone composition described herein has a relatively high resistance to flow. The high viscosity silicone composition described herein is flowable in the presence of an applied stress and behaves more like a shear thinning gel. The silicone composition may be prepared by mixing (a) at least one organopolysiloxane, (b) at least one SiH-containing organopolysiloxane, (c) a thixotropic additive, and (d) a hydrosilyation catalyst.

The thixotropic additive (c) may be any thixotropic additive or agent known in the art. Suitable thixotropic additives include hydrogenated vegetable oils, such as hydrogenated castor oil, hydrogenated soybean oil, hydrogenated canola oil, hydrogenated coconut oil, hydrogenated palm (or palm kernel) oil, hydrogenated sunflower seed oil, and hydrogenated safflower seed oil. Hydrogenated derivatives of known vegetable oils may also be used. The vegetable oils may be converted to hydrogenated vegetable oils through means known in the art. Thixcin® R, a commercially available form of trihydroxystearin from Elementis Specialty Products in the United Kingdom, is reported to have a particle size of less than 44 micron and a melting point of 85-88° C.

Exemplary hydrogenated vegetable oils include hydrogenated castor oil and derivatives of hydrogenated castor oil, such as compounds having the formula (Formula I):

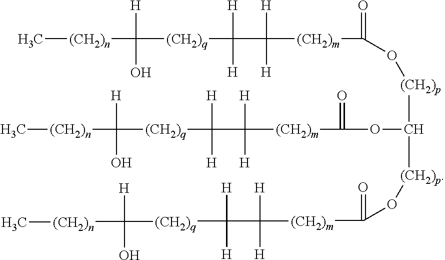

In Formula (I), variables m, n, p, and q are each, independently, an integer ranging from 1-10. For example, p may be an integer ranging from 1-3, for instance 1 or 2; q may be an integer ranging from 1-3, for instance 1 or 2; m may be an integer ranging from 4-10, for instance, an integer ranging from 6-8 or 7; and n may be an integer ranging from 4-10, for instance, an integer ranging from 4-6 or 5.

The thixotropic additive (component (c)) may be present in any amount determined by one skilled in the art that would be sufficient to impart the desired properties of the silicone adhesive gel, described below. Generally, the thixotropic additive may be present in amounts ranging from about 1 to about 15 wt. % based on the total wt. % of the silicone composition. In some embodiments, the thixotropic additive may be present in amounts ranging from about 1 to about 12 wt. % based on the total wt. % of the silicone composition. In still further embodiments, the thixotropic additive may be present in amounts ranging from about 3 to about 12 wt. % based on the total wt. % of the silicone composition. In other embodiments, the thixotropic additive may be present in amounts ranging from about 3 to about 15 wt. % based on the total wt. % of the silicone composition.

Adding the thixotropic additive to the silicone composition may be performed by techniques known in the art. For instance, the thixotropic additive may be added under heat and shear using, for example, a speed mixer and an oven or any change-can type mixer. The additive should be incorporated at a minimum temperature of about 50° C. to build viscosity. The temperature may range from about 50° C. to about 85° C. In some instances, the temperature may range from about 55° C. to about 65° C. A combination of heat and shear facilitates activation of the material, uniform heating, and particle dispersion.

The organopolysiloxane (component (a)) is an aliphatically unsaturated compound. The organopolysiloxane may have an average, per molecule, of one or more aliphatically unsaturated organic groups capable of undergoing hydrosilylation reaction. Alternatively, the organopolysiloxane may have an average of two or more aliphatically unsaturated organic groups per molecule.

The organopolysiloxane has the average formula (Formula II), R¹ₐSiO_{(4-a)/2}, where Formula II may be comprised of the following units: R¹₃SiO_{1/2} (building block M which represents a monofunctional unit); R¹₂SiO_{2/2} (building block D which represents a difunctional unit); R¹₁SiO_{3/2} (building block T which represents a trifunctional unit); or SiO_{4/2} (building block Q which represents a tetrafunctional unit). The number of building blocks (M, D, T, Q) in the organopolysiloxanes may range from 1 to 10,000, for instance from 4 to 1000.

Each of the open bonds from the oxygen atoms, designated as —O—, indicates a position where that building block may be bonded to another building block. Thus, it is through the oxygen atom that a first building block is bonded to a second or subsequent building block, the oxygen bonding either to another silicon atom or one of the R groups in the second or subsequent building block. When the oxygen atom is bonded to another silicon of the second building block, the oxygen atom represented in the first building block acts as the same oxygen atom represented in the second building block, thereby forming a Si—O—Si bond between the two building blocks.

At least one R¹ group is an aliphatically unsaturated group such as an alkenyl group. Suitable alkenyl groups contain from 2 carbon to about 6 carbon atoms and may be, but not limited to, vinyl, allyl, and hexenyl. The alkenyl groups in this component may be located at terminal, pendant (non-terminal), or both terminal and pendant positions. The remaining silicon-bonded organic groups in the alkenyl-substituted polydiorganosiloxane are independently selected from the group consisting of monovalent hydrocarbon and monovalent halogenated hydrocarbon groups free of aliphatic unsaturation. These groups typically contain from 1 carbon to about 20 carbon atoms, alternatively from 1 carbon to 8 carbon atoms and are may be, but not limited to, alkyl such as methyl, ethyl, propyl, and butyl; aryl such as phenyl; and halogenated alkyl such as 3,3,3-trifluoropropyl. In one embodiment, at least 50 percent of the organic groups in the alkenyl-substituted polydiorganosiloxane are methyl. The structure of the alkenyl-substituted polydiorganosiloxane is typically linear however; it may contain some branching due to the presence of trifunctional siloxane units.

Other suitable R¹ groups include, but are not limited to, acrylate functional groups such as acryloxyalkyl groups; methacrylate functional groups such as methacryloxyalkyl groups; cyanofunctional groups; monovalent hydrocarbon groups; and combinations thereof. The monovalent hydrocarbon groups may include alkyl groups such as methyl, ethyl, propyl, isopropyl, n-butyl, s-butyl, t-butyl, pentyl, neopentyl, octyl, undecyl, and octadecyl groups; cycloalkyl groups such as cyclohexyl groups; aryl groups such as phenyl, tolyl, xylyl, benzyl, and 2-phenylethyl groups; halogenated hydrocarbon groups such as 3,3,3-trifluoropropyl, 3-chloropropyl, dichlorophenyl, and 6,6,6,5,5,4,4,3,3-nonafluorohexyl groups; and combinations thereof. The cyano-functional groups may include cyanoalkyl groups such as cyanoethyl and cyanopropyl groups, and combinations thereof.

R¹ may also include alkyloxypoly(oxyalkyene) groups such as propyloxy(polyoxyethylene), propyloxypoly(oxypropylene) and propyloxy-poly(oxypropylene)-co-poly(oxyethylene) groups, halogen substituted alkyloxypoly(oxyalkyene) groups such as perfluoropropyloxy(polyoxyethylene), perfluoropropyloxypoly(oxypropylene) and perfluoropropyloxy-poly(oxypropylene) copoly(oxyethylene) groups, alkenyloxypoly(oxyalkyene) groups such as allyloxypoly(oxyethylene), allyloxypoly(oxypropylene) and allyloxy-poly(oxypropylene) copoly(oxyethylene) groups, alkoxy groups such as methoxy, ethoxy, n-propoxy, isopropoxy, n-butoxy and ethylhexyloxy groups, aminoalkyl groups such as 3-aminopropyl, 6-aminohexyl, 11-aminoundecyl, 3-(N-allylamino)propyl, N-(2-aminoethyl)-3-aminopropyl, N-(2-aminoethyl)-3-aminoisobutyl, p-aminophenyl, 2-ethylpyridine, and 3-propylpyrrole groups, hindered aminoalkyl groups such as tetramethylpiperidinyl oxypropyl groups, epoxyalkyl groups such as 3-glycidoxypropyl, 2-(3,4,-epoxycyclohexyl)ethyl, and 5,6-epoxyhexyl groups, ester functional groups such as acetoxymethyl and benzoyloxypropyl groups, hydroxyl functional groups such as hydroxy and 2-hydroxyethyl groups, isocyanate and masked isocyanate functional groups such as 3-isocyanatopropyl, tris-3-propylisocyanurate, propyl-t-butylcarbamate, and propylethylcarbamate groups, aldehyde functional groups such as undecanal and butyraldehyde groups, anhydride functional groups such as 3-propyl succinic anhydride and 3-propyl maleic anhydride groups, carboxylic acid functional groups such as 3-carboxypropyl, 2-carboxyethyl, and 10-carboxydecyl groups, metal salts of carboxylic acids such as zinc, sodium, and potassium salts of 3-carboxypropyl and 2-carboxyethyl groups, and combinations thereof.

Particular examples of organopolysiloxanes include polydimethysiloxane-polymethylvinylsiloxane copolymers, hexenyldimethylsiloxy-terminated polydimethylsiloxane-polymethylhexenylsiloxane copolymers, hexenyldimethylsiloxy-terminated polydimethylsiloxane polymers, vinyldimethylsiloxy-terminated polydimethylsiloxane polymers, vinyl or hexenyldimethylsiloxy-terminated poly(dimethylsiloxane-silicate) copolymers, mixed trimethylsiloxy-vinyldimethylsiloxy terminated poly(dimethylsiloxane-vinylmethylsiloxane-silicate) copolymers, vinyl or hexenyldimethylsiloxy terminated poly(dimethylsiloxane-hydrocarbyl) copolymers, derivatives thereof, and combinations thereof. Functional groups may be present at any point in the organopolysiloxane, for example, in the middle of the polymer or as an endgroup(s). Typical functional groups, such as diorgano-, —OH, -vinyl, -hexenyl, -epoxy, and -amine may be used in the organopolysiloxanes contemplated herein. End groups such as Me₃, Ph₂Me, Me₂Ph may or may not be present in the organopolysiloxane.

The SiH-containing organopolysiloxane (component (b)) is also known in the art as described, for example, in U.S. Pat. No. 3,983,298. The hydrogen atoms in this component may be located at terminal, pendant (non-terminal), or both terminal and pendant positions. The remaining silicon-bonded organic groups in this component are independently selected from the group consisting of monovalent hydrocarbon and monovalent halogenated hydrocarbon groups free of aliphatic unsaturation. These groups typically contain from 1 carbon to about 20 carbon atoms, alternatively from 1 carbon to 8 carbon atoms, and are exemplified by, but not limited to, alkyl such as methyl, ethyl, propyl, and butyl; aryl such as phenyl; and halogenated alkyl such as 3,3,3-trifluoropropyl. In one embodiment, at least 50 percent of the organic groups in the organosiloxane containing silicon-bonded hydrogen atoms are methyl. The structure of the organosiloxane containing silicon-bonded hydrogen atoms is typically linear however; it may contain some branching due to the presence of trifunctional siloxane units.

The SiH-containing organopolysiloxane has the average formula (Formula III), R²ₐSiO_{(4-a)/2}, where Formula III may be comprised of the following units: R²₃SiO_{1/2} (or building block M); R²₂SiO_{2/2} (or building block D); R²₁SiO_{3/2} (or building block T); or SiO_{4/2} (or building block Q). The number of building blocks (M, D, T, Q) in the organopolysiloxanes may range from 1 to 10,000, for instance from 4 to 1000. R¹ and R² are different because at least one R¹ has to be C═C and at least one R² has to be H.

Each of the open bonds from the oxygen atoms, designated as —O—, indicates a position where that building block may be bonded to another building block. Thus, it is through the oxygen atom that a first building block is bonded to a second or subsequent building block, the oxygen bonding either to another silicon atom or one of the R groups in the second or subsequent building block. When the oxygen atom is bonded to another silicon of the second building block, the oxygen atom represented in the first building block acts as the same oxygen atom represented in the second building block, thereby forming a Si—O—Si bond between the two building blocks.

In one embodiment, the number of building blocks (M, D, T, Q) in the SiH-containing organopolysiloxanes is from 1 to 1000. The SiH-containing organopolysiloxanes must contain at least one M, at least one D, or at least one T building block. In other words, the SiH-containing organopolysiloxanes cannot contain all Q building blocks. If there is only one building block, it can only be chosen from M, D, or T.

The SiH-containing organopolysiloxane may be a linear or cyclic compound containing from 1-10,000 (for instance, 1-1000, 1-200, or 1-100) of any combination of the following M, D, T, and Q building blocks. Examples of the SiH-containing materials described by Formula III that are useful in the methods described herein include oligomeric and polymeric organosiloxanes, such as (i) cyclic compounds containing 3-25 D building blocks (for instance, 3-10 or 4-6 D building blocks); or (ii) linear compounds containing two M building block that act an end blocks, and 2-10,000 D building blocks (for instance, 2-1000, 2-200, 10-100, 50-80, 60-70, 2-20, or 5-10) between the end blocks. Linear SiH-containing organopolysiloxanes may be particularly useful in some embodiments, for example, those containing combination(s) of pendant and terminal SiH groups.

Various other compounds or additives may be added to the silicone composition. For example, the gel may contain one or more silicon-based resins, such as a hydroxy-substituted siloxane resin(s). Hydroxy-substituted siloxane resin(s) increase the adhesion of the gel to, for example, medical substrates and skin.

The hydroxy-substituted siloxane resin(s) comprise R3SiO1/2 units (M units) and SiO4/2 units (Q units) wherein each R is independently a linear, branched or cyclic hydrocarbon group having 1-20 carbon atoms. R can be unsubstituted or substituted with halogen atoms. Each R can be identical or different, as desired. The hydrocarbon group of R can be exemplified by alkyl groups such as methyl, ethyl, propyl, butyl, hexyl, octyl, 3,3,3-trifluoropropyl, chloromethyl, and decyl, alkenyl groups such as vinyl and hexenyl, cycloaliphatic groups such as cyclohexyl, aryl groups such as phenyl, tolyl, and xylyl, chlorophenyl, and aralkyl groups such as benzyl, styryl and alpha-methylstyryl. Alternatively, each R group is an independently selected alkyl or alkenyl group comprising 1 to 8 carbon atoms or aryl group comprising 6 to 9 carbon atoms. Alternatively, each R group is independently selected from methyl and vinyl.

If an alkenyl group is present in the hydroxy-substituted siloxane resin(s), typically the mole % of R groups present as alkenyl groups is less than about 10%, alternatively less than about 5%. For example, if the resin contains vinyl groups, typically they are present in an amount of less than about 5 wt. % of the resin solids, alternatively less than about 2.5 wt. % of the resin solids, alternatively about 1.5-2 wt. % of the resin solids.

The molar ratio of R3SiO1/2 units (M units) to SiO4/2 units (Q units) can be from about 0.6:1 to 4:1. Alternatively, the molar ratio of M:Q can be from about 0.6:1 to 1.9:1. Alternatively, the molar ratio of M:Q can be from about 0.6:1 to 1.0:1. The resins can also contain triorganosiloxy units (T units), for example about 0.5 to 1 triorganosiloxy group for every SiO4/2 unit, alternatively about 0.6 to 0.9 triorganosiloxy group for every SiO4/2 unit. It should be noted that more than one resin could be included in the present invention. In this case, at least one of the resins should have the silanol content as described below but, by the same token, one could have the silanol capped so that there is substantially no silanol present.

It should also be noted that other resins can also be added to the silicone composition contemplated herein. For example, organic resins could be added if desired. In one embodiment, for example, a vinyl-functional organic resin can be added.

In one embodiment, a majority of all R groups in R3SiO1/2 are methyl and the total number of R groups in R3SiO1/2 are methyl and the total number of R groups that have olefinic unsaturation is no more than about 0.5% of all R groups. In another embodiment, substantially all of the R groups in R3SiO1/2 are methyl. In another embodiment, substantially all of the R groups in R3SiO1/2 are substantially free of olefinic unsaturation. In yet another embodiment, two resins are included—one in which substantially all of the R groups in R3SiO1/2 are methyl and the other in which about 3.5 to 4 mole % of the R groups in R3SiO1/2 are vinyl and substantially all of the remaining R groups are methyl. The resins also contain silicon-bonded hydroxyl groups ranging from about 0.01 up to about 5 wt. % of the resin, alternatively from about 1 to about 5 wt. % of the resin.

The organopolysiloxane (component (a)) and the SiH-containing organopolysiloxane (component (b)) may be present in any amount determined by one skilled in the art that would be sufficient to impart the desired properties of the silicone adhesive gel described herein. Generally, the SiH-containing organopolysiloxane to organopolysiloxane ratio ranges from about 0.8 to about 0.9.

If desired, other components can be added to the silicone composition including, but not limited to, fillers, pigments, low-temperature cure inhibitors, additives for improving adhesion, chain extenders, pharmaceutical agents, drugs, cosmetic agents, natural extracts, fluids or other materials conventionally used in gels, silicone fluids, silicone waxes, silicone polyethers, and rheology modifiers such as thickening agents or additional thixotropic agents.

To form the silicone composition, the components (components (a), (b) and (c)) are combined in the presence of a hydrosilyation catalyst (d). Suitable hydrosilyation catalysts include platinum catalysts such as chloroplatinic acid, alcohol solutions of chloroplatinic acid, dichlorobis(triphenylphosphine)platinum(II), platinum chloride, platinum oxide, complexes of platinum compounds with unsaturated organic compounds such as olefins, complexes of platinum compounds with organosiloxanes containing unsaturated hydrocarbon groups, such as Karstedts catalyst (i.e. a complex of chloroplatinic acid with 1,3-divinyl-1,1,3,3-tetramethyldisiloxane) and 1,3-diethenyl-1,1,3,3-tetramethyldisiloxane, and complexes of platinum compounds with organosiloxanes, wherein the complexes are embedded in organosiloxane resins. For example, a hydrosilyation catalyst may be a 0.5% platinum containing platinum-divinyltetramethyldisiloxane, a complex that is commercially available from Dow Corning Corporation of Midland, Mich. The hydrosilyation catalyst may be added to the composition in an amount sufficient to provide, for example, 1 to 10 ppm of platinum based on the total weight of the silicone composition. Upon combining, the silicone composition is cured to form a silicone adhesive gel.

The silicone composition exhibits (i) viscosity ranging from about 7000 cP to about 5,000,000 cP and (ii) shear thinning behavior, as determined by the rheological profile. The resulting silicone gel adhesive exhibits (i) adhesiveness ranging from about 0.2 N to about 4 N, as determined by the peel adhesion test and (ii) cohesive strength, as determined by the peel adhesion test.

Viscosity of the silicone composition may be determined using a Brookfield viscometer or with a Helipath stand. The Brookfield viscometer measures viscosity by measuring the force required to rotate a spindle in fluid. The high viscosity silicone compositions contemplated herein have a viscosity ranging from about 7000 cP to about 5,000,000 cP. This viscosity range provides the silicone with viscosity that allows it hold a pattern when applied on a substrate without significantly absorbing into the substrate. Alternatively, the viscosity ranges from about 15,000 cP to about 5,000,000 cP, or from about 20,000 cP to about 5,000,000 cP. The application viscosity depends on the amount and type of shear applied.

In accordance with the Standard Test Method for Apparent Viscosity of Adhesives Having Shear-Rate-Dependent Flow Properties, ASTM-2556-93a (2005), the rheological properties of the silicone adhesive gel may be measured. Shear thinning or pseudoplastic behavior is the behavior exhibited when viscosity decreases with an increasing rate of shear stress. By analyzing the rheological profile of the silicone adhesive gel, it can be determined whether or not the silicone adhesive gel will exhibit shear thinning behavior.

Adhesion may be determined by peel adhesion tests. In accordance with the International Standard for Peel Adhesion of Pressure Sensitive Tape, PSTC-101 (issued October 2000 and last revised May 2007), peel adhesion tests show the pull-off adhesion strength of pressure sensitive tapes. For the purposes of this application, an adhesive gel that has low peel adhesion properties does not possess adhesiveness. When the adhesiveness drops much below 0.2 N, it does not possess a sufficient amount of adhesiveness to act as an adhesive gel, for instance to adhere to the outside layer of a wound; when the adhesiveness increases much above 4 N, the application and subsequent removal of the adhesive gel from the wound can become problematic or discomforting to the patient. Alternatively, the adhesiveness ranges from about 1.5 N to about 3 N; alternatively, from about 1.7 to about 3 N.

Cohesive strength may be determined by peel adhesion tests. In accordance with the International Standard for Peel Adhesion of Pressure Sensitive Tape, PSTC-101 (issued October 2000 and last revised May 2007), peel adhesion tests show the pull-off adhesion strength of pressure sensitive tapes. For the purposes of this application, an adhesive gel that does not remain intact during the test does not possess cohesive strength.

It is contemplated that the silicone composition may be prepared as a multiple part (e.g., 2 part) composition, for example, when the composition will be stored for a long period of time before use. In the multiple part composition, the catalyst is stored in a separate part from any ingredient having a silicon bonded hydrogen atom, for example ingredient (b), and the various parts are combined shortly before use of the composition.

The silicone gel adhesive compositions described herein may be used as the skin-facing layer of a medical device or wound dressing. In addition to the silicone gel adhesive composition, the medical dressing contains an absorbable or porous substrate. The absorbable substrate may be any material known to those of skill in the art capable of at least partially absorbing the exudate from the wound. Absorbable substrates include, but are not limited to, the following materials: foams (e.g., polyurethane and/or polymer foams), synthetic sponges, natural sponges, silks, keratins (e.g., wool and/or camel hair), cellulosic fibers (e.g., wood pulp fibers, cotton fibers, hemp fibers, jute fibers, and/or flax fibers), rayon, acetates, acrylics, cellulose esters, modacrylics, polymers, super-absorbent polymers (e.g., polymers capable of absorbing approximately 10 times their weight or greater), polyamides, polyesters, polyolefins, polyvinyl alcohols, and/or other materials. Combinations of one or more of the above-listed materials may also be used as the absorbable or porous substrate.

The silicone gel adhesive compositions described herein may also be used as the skin-facing layer in various applications where suitable skin-facing adhesive materials are desired. Representative examples of additional skin-facing uses of the adhesive compositions described herein are in athletic apparel such as biking shorts and feminine hygiene products.

Other additives or agents commonly added to medical dressings may also be included in the dressing. For instance, the medical dressing may also include agents that provide a pain-relieving effect, antiseptic effect, help sterility, speed healing. The agents may be added separately or impregnated into the silicone composition, absorbable substrate, or other component of the medical dressing. For instance, dressings are commonly impregnated with antiseptic chemicals, such as in boracic lint. In one embodiment, the medical dressing contains silver particles, either suspended in the adhesive gel or otherwise impregnated into the dressing, which can be used to impart antimicrobial properties into the dressing.

A medical dressing, as known to those of skill in the art, is an adjunct used by a person for application to a wound to promote healing and/or prevent further harm. A medical dressing is designed to be in direct contact with the wound, although, for the purposes of this application, direct contact on all areas of the wound is not necessary. Among other purposes, a medical dressing is designed to (a) stem bleeding and help to seal the wound to expedite the clotting process; (b) absorb exudate by soaking up blood, plasma and other fluids exuded from the wound; (c) ease pain of the wound; (d) debride the wound by removing the slough and foreign objects from the wound; (e) protect the wound from infection and mechanical damage; and (f) promote healing through granulation and epithelialization. A medical dressing comprising the silicone gel adhesive composition described herein, like other medical dressings, is designed to accomplish one or more of these design objectives.

It is also desirable for the medical dressing to retain a sufficient amount of moisture without retaining too much moisture, which can lead to an excessively wet environment for the wound which promotes the growth of bacteria, thus leading to wound maceration or other ailments. Balancing the moisture vapor is one way to gauge whether the dressing contains an appropriate amount of moisture. Other measures may also be used.

Making the silicone adhesive layer of the medical dressing discontinuous is one way to promote a balanced moisture vapor. Medical dressings can be made discontinuous in various ways, for instance by utilizing a perforated carrier material to create a path for exudate to pass through to the absorbent pad. One example of such a perforation process involves making small holes in the polyurethane carrier film to which the silicone composition is applied, then blowing air through the holes or using an ultrasonic device to open up the holes in the silicone layer while the composition cures.

Another means of making the silicone layer of a medical dressing discontinuous involves applying the silicone composition on the substrate in a pattern so that the pattern naturally creates discontinuity in the areas on the substrate that are not coated with the silicone composition. Similar to creating a carrier material with perforations, applying the discontinuous (or semi-continuous) pattern on the substrate creates a coating with void areas that allow exudate to pass through to the substrate to be absorbed. Any predetermined pattern that creates the void areas is sufficiently discontinuous for these purposes. The discontinuity of the pattern also enables an avenue for the moisture to be released from the wound, promoting a balanced moisture vapor. Accordingly, one contemplated embodiment relates to a silicone composition that has the ability to be pattern coated on a substrate such as an absorbable substrate; another embodiment relates to a medical dressing containing a substrate such as an absorbable substrate pattern coated with a silicone composition; and yet another embodiment to a method of preparing a medical dressing comprising the step of coating the silicone composition onto a substrate such as an absorbent substrate in a predetermined pattern.

The silicone composition may be applied to the substrate using any means known in the art, for instance through a screen printing or stenciling process. In the screen printing process, a screen or woven mesh is typically placed atop of the substrate, where the mesh contains a design that provides for an open area to transfer. The operator uses a roller or a squeegee to apply the silicone composition by pressing the gel through the mesh onto the substrate as the squeegee or roller is pushed to the rear of the screen. The thickness of the silicone composition is generally proportional to the thickness of the mesh or stencil. Thus, the thickness of the silicone composition that is applied or coated onto the substrate may be controlled by the screen or mesh that is used in the application process. A typical thickness of the silicone composition ranges from about 3 mil (76.2 μm) to about 20 mil (508 μm). In other instances, the typical thickness of the silicone composition may range from about 5 mil (127 μm) to about 15 mil (381 μm). In further instances, the typical thickness of the silicone composition may range from about 8 mil (203.2 μm) to about 12 mil (304.8 μm). Other thicknesses can also be used, depending on the desired result. As the squeegee moves toward the rear of the screen, the tension of the mesh pulls the mesh up away from the substrate, leaving the silicone composition on the substrate surface.

There are three common types of screen-printing presses: the “flat-bed,” “cylinder,” and “rotary,” with the rotary press being the most common. These processes can be used to apply the silicone composition described herein onto a substrate such as an absorbable substrate. Any screen-printing press may be used in these processes. In a typical rotary screen printing, a passing web is pressed by a press roller against a heated engraved roller, the cavities of which are filled by a liquid that is applied by a doctor blade. The applicator unit is a heated trough that is sealed off against the engraved roller by a spring steel doctor blade. Via pressure of the engraved roller against the substrate, the material is transferred onto the web and a patterned coating, which conforms with the configuration of the engraved roller, is achieved. Processes such as, but not limited to, reverse-offset and gravure-offset rotary screen printing techniques may be used to apply the silicone composition described herein onto the absorbable substrate.

Automated dispensers, such as those manufactured by Graco, Inc. in Minneapolis, Minn., may also be used to apply the silicone composition described herein onto the substrate. Automated dispensing units, such as those sold by Graco, Inc., offer a precise, positive displacement metering using double-acting cylinders and fluid inlet pressure to continuously reciprocate two connected cylinders. As the major volume cylinder (base) and minor volume cylinder (catalyst) reciprocate, they positively displace the two material components on ratio to the outlet ports. Static mixers are incorporated into the system to deliver a homogeneous mix of base and catalyst.

One of the unique benefits of the silicone composition is its ability to be pattern coated directly onto the substrate in a manner where the pattern of the coating is maintained upon application. It is believed that the combination of properties exhibited by the silicone composition, including the adhesion, viscosity, cohesive strength, and rheology discussed above enable this feature. Advantageously, the silicone composition does not penetrate most absorbent substrates, or only penetrates the substrate minimally, while staying on the surface and maintaining the pattern. As discussed above, maintaining the pattern to create the voids provides the desired discontinuity, which in turn, allows the exudate to pass through to the substrate and promotes a balanced moisture vapor.

Yet another embodiment relates to a silicone composition having high-density particles suspended in the silicone composition, the silicone composition comprising: (a) at least one organopolysiloxane, (b) at least one SiH-containing organopolysiloxane, and (c) about 0.1 to about 3 wt. % of a hydrogenated vegetable oil. The silicone composition is capable of suspending the high-density particles in the silicone composition.

Various hydrogenated vegetables oils, such as those discussed above, are suitable for use in this embodiment. Exemplary hydrogenated vegetable oils include hydrogenated castor oil and derivatives thereof, including compounds having the formula (Formula I):

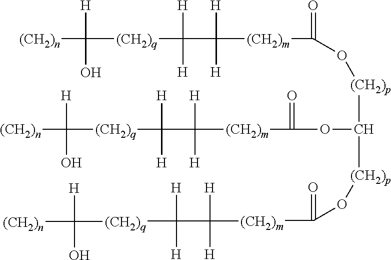

where variables m, n, p, and q are each, independently, an integer ranging from 1-10. For example, p may be an integer ranging from 1-3, for instance 1 or 2; q may be an integer ranging from 1-3, for instance 1 or 2; m may be an integer ranging from 4-10, for instance, an integer ranging from 6-8 or 7; and n may be an integer ranging from 4-10, for instance, an integer ranging from 4-6 or 5.

In some embodiments, utilizing about 0.1 to about 3 wt. % of a hydrogenated vegetable oil provides the desired consistency to the silicone composition to suspend high-density particles. In still further embodiments, the desired consistency can be provided to the silicone composition to suspend high-density particles by utilizing about 0.25 to about 2 wt. % of a hydrogenated vegetable oil provides. In other embodiments, the desired consistency can be provided to the silicone composition to suspend high-density particles by utilizing about 0.5 to about 1.5 wt. % of a hydrogenated vegetable oil.

The high-density particles include any particles that can be difficult or problematic to suspend in liquid or gel-like compositions. Metal particles or compounds that contain metal commonly have a high density. Besides metal particles, other high-density particles such as certain high-density fillers, salts, powdered pigments, hydrophilic compositions, actives, pharmaceuticals, and additives can also be suspended.

Notable metal particles that can be suspended in the silicone composition include silver-containing particles. These silver-containing particles may be in the form of silver compounds, for example silver salts, silver carboxylates, organosilver compounds, silver sulfates, silver alkyl sulfates, silver aryl sulfates, silver alkyl sulfonates, and silver arylsulfonates.

When metal particles are used as the high-density particles, the metal salt concentration in the silicone composition typically ranges from about 1 wt. % to about 10 wt. % based on the total wt. % of the silicone composition. Metals in their metallic form may also be used in the silicone composition.

Like the high viscosity, shear-thinning silicone composition discussed above, the silicone composition having high-density particles suspended in it may be utilized as part of a medical dressing. Medical dressings containing silicone compositions with silver particles are particularly advantageous because of the well-known antimicrobial properties of the silver. When the silver particles become ionized, typically through contact with the moisture from the exudate, the particles become activated and can then provide antimicrobial effects. Other benefits of using silver particles in medical dressings have been well-recognized in the art.

Because silicon is hydrophobic and many silicon-based compositions are hydrophobic, a hydrophilic additive may be included in the silicone composition to assist in drawing the moisture into the gel to activate the silver ions. Suitable hydrophilic additives include compounds such as silicon polyethers, polyethylene oxides, PVP, PEG, sulfoisophthalic acid co-polymers, amine compounds, sugars, alcohols, cellulosic materials, polymers having side chains of carboxyl groups or hydroxyl groups, polyacrylic acids, carboxylic acids, salts of carboxylic acids, amides, urethanes, compounds having oxyalkylenated groups, and the like.

Metal particles, because of the weight of the metal, are prone to settle towards the bottom of the composition over a period of time. For example, silver is roughly five times the weight of silicon. While long-term suspension, for instance, suspending metal particles long periods of time in the range of 6 to 12 months, is often not necessary, suspending the metal particles in the gel for at least 24 hours is advantageous. Having a composition with metal particles suspended for 24 hours allows the end user to agitate the silicone composition prior to application. For instance, when the silicone composition is part of a medical dressing, the gel containing the metal particles can be agitated, i.e. shaken up, to re-suspend the particles in the gel (for at least 24 hours) so that they are uniformly dispersed and can be effective upon application of the medical dressing to a wound.

### EXAMPLES

### Example 1

7.5 wt. % of hydrogenated castor oil, used as a thixotropic additive, was added to both parts of a two-part formulation having the following composition using heat and shear to incorporate and activate the thixotropic additive in each part:

TABLE A
Silicone Composition

| Material | Wt. % |
|---|---|
| dimethylvinylsiloxy-terminated | 35.485 |
| polydimethylsiloxane with 450 cP viscosity | |
| alkenyl-substituted polydiorganosiloxane | 24.50 |
| with 2,000 cP viscosity | |
| dimethylvinylsiloxy-terminated | 10.00 |
| polydimethylsiloxane with 55,000 cP | |
| viscosity | |
| platinum hydrosilylation catalyst | 0.20 |
| dimethylhydrogen-terminated | 29.20 |
| polydimethylsiloxane | |
| methyl hydrogen, dimethyl copolymer | 0.60 |
| tetramethyltetravinyl cyclotetrasiloxane | 0.015 |

### Preparation 1

### Planetary Disperser Mixer (7.5% Hydrogenated Oil)

The material components for Part A were added to a jacketed pot of a planetary disperser mixer. An oil bath feeding the jacketed pot was used to heat the material within the mixer. To incorporate the thixotropic additive with the silicone composition, the mixing was performed slowly at first: 23 RPM for 3 minutes, followed by 46 RPM for 2 minutes, with the disperser turned off. The material still exhibited a lower viscosity at this point. The oil bath feeding the jacketed pot was set to 80° C. to slowly heat the mixture of gel adhesive and thixotropic additive. The material was then mixed at a fast rate for 5 minutes to provide shear, with the planetary mixer set to 93 RPM and the disperser set to 3420 RPM. An increase in viscosity was observed, but the material still flowed. The speed was then reduced to 23 RPM on the planetary and 0 RPM on the disperser, and the material was mixed until the temperature of the material reached 65° C. The final material was very viscous and exhibited non-flowing behavior. The same procedure was then repeated for Part B of the formulation. Both parts were then combined in a 1:1 ratio and mixed in a dual asymmetric centrifuge (DAC) mixer for 3×16 seconds to combine them into a homogenous fluid.

The material was then pattern coated on polyester, non-woven fabric, and foam and cured at 130° C. for 4 minutes. The silicone composition was cured in place, keeping the open patterned design. Patterns were achieved using stencils and screens.

Samples for release and adhesion were prepared by mixing the two parts of the formulation with the thixotropic additive in a dual asymmetric centrifuge mixer. The adhesive with the thixotropic additive was coated to approximately 0.25 mm thickness on a polyester substrate using a table top coater and 0.38 mm shims. The coated substrate was cured in an oven for 4-5 minutes at 130° C. After removing the coated substrate from the oven, it was immediately covered with LDPE diamond embossed release liner using a 15 lb (6.8 kg) rubber coated roller. The sample was allowed a minimum of 16 hours to equilibrate prior to testing. The coated substrate was cut into 2.54 cm strip with a minimum of 12.7 cm in length.

Release and adhesion were evaluated using a Texture Analyzer with the Self Tightening Roller Grips attachment with the clamps set 25 mm apart.

Release

For release testing, the release liner was secured in the bottom clamp and the adhesive coated polyester was placed in the top clamp. The clamps were pulled apart at 10 mm/s for 130 mm. The resultant force to pull the release liner from the adhesive coated polyester was averaged over 10 cm (excluding the first 2 cm and last 1 cm of the 13 cm pull) and measured in Newtons per centimeter (N/cm). The final release value is the average of 5 test strips.

Adhesion

For adhesion testing, the release liner was removed from the coated polyester and the test strip was adhered to the frosted side of a 1.5 in×7 in (3.8 cm×17.8 cm) strip of polycarbonate (Lexan GE Product No. 8813-112D) using a 5 lb (2.3 kg) rubber coated roller making one stroke forward and one stroke back at a rate of 1 in/sec (2.5 cm/sec). The sample was allowed to equilibrate for 30 minutes. The polycarbonate was secured in the bottom clamp and the adhesive coated polyester was placed in the top clamp. The clamps were pulled apart at 10 mm/s for 130 mm. The resultant force to pull the polycarbonate from the adhesive coated polyester was averaged over 10 cm (excluding the first 2 cm and last 1 cm of the 13 cm pull) and measured in Newtons per centimeter (N/2.5 cm). The final release value is the average of 5 test strips. Release was 0.06 N/2.5 cm after 1 day and was 0.09 N/2.5 cm after 7 days. Adhesion was 2.81 N/2.5 cm after 1 day and was 1.96 N/2.5 cm after 7 days. The testing was performed on samples aged in a temperature controlled chamber set at 40° C.

Cohesion

Cohesion was evaluated during the adhesion testing by determining how much adhesive remained on the polycarbonate. Measurements of cohesive failure were made by estimating the percentage of adhesive remaining on the polycarbonate surface. There was no cohesive failure.

Viscosity The viscosity of Parts A and B was measured on a Brookfield DV-II+ Viscometer with a Helipath Stand (Model D). The viscosity was measured with spindle T-E at 2.5 rpm. The samples were vacuum de-aired prior to testing. Ten data points were acquired during the initial down cycle. The reported viscosity was an average of the ten data points. Part A had a viscosity of 292,000 cP. Part B had a viscosity of 204,000 cP.

Rheoloqy

The rheology of Parts A and B was evaluated by performing a frequency sweep on a strain controlled rheometer, TA Instrument ARES, across a frequency range of 0.01 rad/s to 100 rad/s at 0.1% strain and 30° C. (gap=1.5 mm). The rheology results are summarized in Tables B and C below.

TABLE B
Rheology of Part A

| Freq | Eta* | G′ | G″ | | Strain | Time |
|---|---|---|---|---|---|---|
| (rad/s) | (P) | (dyn/cm²) | (dyn/cm²) | tan _delta | (%) | (s) |
| 0.01 | 1,977,832 | 19,430 | 3,697 | 0.190 | 0.099 | 321 |
| 0.032 | 694,439 | 21,789 | 2,737 | 0.126 | 0.099 | 2419 |
| 0.1 | 238,020 | 23,683 | 2,381 | 0.101 | 0.099 | 2752 |
| 0.316 | 66,427 | 20,844 | 2,601 | 0.125 | 0.099 | 2858 |
| 1 | 22,232 | 22,091 | 2,494 | 0.113 | 0.099 | 2907 |
| 3.162 | 7,649 | 23,874 | 3,889 | 0.163 | 0.100 | 2927 |
| 10 | 2,761 | 26,764 | 6,783 | 0.253 | 0.100 | 2948 |
| 31.623 | 1,138 | 33,901 | 12,118 | 0.357 | 0.099 | 2963 |
| 100 | 473 | 40,406 | 24,635 | 0.610 | 0.075 | 2970 |

TABLE C
Rheology of Part B

| Freq | Eta* | G′ | G″ | | Strain | Time |
|---|---|---|---|---|---|---|
| (rad/s) | (P) | (dyn/cm²) | (dyn/cm²) | tan _delta | (%) | (s) |
| 0.01 | 997,508 | 9,670 | 2,448 | 0.253 | 0.099 | 320 |
| 0.032 | 362,595 | 11,343 | 1,674 | 0.148 | 0.099 | 2216 |
| 0.1 | 126,307 | 12,562 | 1,313 | 0.105 | 0.099 | 2549 |
| 0.316 | 42,185 | 13,250 | 1,549 | 0.117 | 0.099 | 2655 |
| 1 | 13,929 | 13,779 | 2,039 | 0.148 | 0.100 | 2693 |
| 3.162 | 4,854 | 15,072 | 2,904 | 0.193 | 0.100 | 2705 |
| 10 | 1,792 | 17,117 | 5,304 | 0.310 | 0.100 | 2713 |
| 31.623 | 724 | 20,503 | 10,157 | 0.495 | 0.099 | 2721 |
| 100 | 349 | 28,087 | 20,716 | 0.738 | 0.073 | 2729 |

To evaluate the rheology of the crosslinked material (Parts A and B combined), equal Parts A and Parts B were mixed using a dual asymmetric centrifuge mixer. The combined material was mixed for 16 seconds then mixed with a spatula. The material was then placed back in the dual asymmetric centrifuge mixer and mixed two more times for 16 seconds each (spatula mixed after second mix). The combined parts of A and B were cured on a strain controlled rheometer, TA Instrument ARES, at 130° C. for 20 minutes. After cooling, the rheology was evaluated across a frequency range of 0.01 rad/s to 100 rad/s (log scale—2 points per decade) at 3.0% strain and 30° C. (gap=1.4 mm). The rheology of cured Parts A and B is shown in Table D below.

TABLE D
Rheology of Cured Parts A and B

| Freq | Eta* | G′ | G″ | | Strain | Time |
|---|---|---|---|---|---|---|
| (rad/s) | (P) | (dyn/cm²) | (dyn/cm²) | tan _delta | (%) | (s) |
| 0.010 | 895,107 | 7,921 | 4,168 | 0.526 | 2.996 | 320 |
| 0.032 | 420,265 | 11,152 | 7,229 | 0.648 | 2.993 | 1430 |
| 0.100 | 211,055 | 16,748 | 12,843 | 0.767 | 2.992 | 1763 |
| 0.316 | 111,391 | 26,747 | 22,921 | 0.857 | 2.991 | 1869 |
| 1.000 | 60,563 | 44,736 | 40,824 | 0.913 | 2.987 | 1904 |
| 3.162 | 33,500 | 77,120 | 72,628 | 0.942 | 2.984 | 1916 |
| 10.000 | 18,619 | 136,981 | 126,106 | 0.921 | 2.980 | 1924 |
| 31.623 | 10,152 | 245,663 | 206,673 | 0.841 | 2.976 | 1932 |
| 100.000 | 5,263 | 426,775 | 308,020 | 0.722 | 2.926 | 1939 |

### Example 2

7.5% hydrogenated castor oil, used as a thixotropic additive, was added to both parts of a two-part formulation having the following composition using heat and shear to incorporate and activate the thixotropic additive in each part.

TABLE E
Resin-Loaded Adhesive Composition

| Material | Wt. % |
|---|---|
| dimethylvinylsiloxy-terminated | 52.5 |
| polydimethylsiloxane with 450 cP viscosity | |
| alkenyl-substituted polydiorganosiloxane resin | 36.4 |
| platinum hydrosilylation catalyst | 0.400 |
| methyl hydrogen, dimethyl copolymer | 0.020 |
| dimethylhydrogen-terminated polydimethylsiloxane | 7.200 |
| dimethyl siloxane, dimethylvinylsiloxy-terminated | 3.4 |
| with 2,000 cP viscosity | |

### Preparation 2

### Dual Asymmetric Centrifuge Mixer (7.5% Hydrogenated Oil)

The material components for Part A were added to a 100 gram mixer cup. The cup was placed in a dual asymmetric centrifuge mixer, then mixed for 15 seconds at 3500 RPM.

The material was then mixed with a spatula and placed back in the dual asymmetric centrifuge mixer and mixed an additional two times at 3500 RPM for 15 seconds. The mixture was then placed in a forced-air oven set to 70° C. After 30 minutes, the cup was removed from the oven and placed in a dual asymmetric centrifuge mixer and mixed for 15 seconds at 3500 RPM. An increase in viscosity was observed, but the material still flowed (material temperature was approx. 55° C.). The cup was placed back in the oven set to 70° C. for approximately 30 minutes. The temperature of the material was approximately 60° C. The cup was again placed in a dual asymmetric mixer and mixed for 15 seconds at 3500 RPM, removed and spatula mixed. The cup was returned to the 70° C. oven for two additional 30 minutes intervals, each followed by 15 seconds of mixing at 3500 RPM. The final material was very viscous and exhibited non-flowing behavior. The same procedure was then repeated for Part B of the formulation. Both parts were then combined in a 1:1 ratio and mixed in a dual asymmetric centrifuge (DAC) mixer for 3×16 seconds to combine them into a homogenous fluid.

The material was then pattern coated on polyester, non-woven fabric, and foam and cured at 130° C. for 4 minutes, as performed in Example 1.

Release and Adhesion

Samples for release and adhesion were prepared and evaluated as specified in Example 1. Release was 2.44 N/2.5 cm after 1 day and was 2.73 N/2.5 cm after 7 days at room temperature. Adhesion was 3.95 N/2.5 cm after 1 day and was 3.29 N/2.5 cm after 7 days at room temperature.

Viscosity

The viscosity of Parts A and B was measured on a Brookfield DV-II+Viscometer with a Helipath Stand (Model D) with spindle T-E at 2.5 rpm under conditions as specified in Example 1. Part A had a viscosity of 692,000 cP. Part B had a viscosity of 506,000 cP.

Rheoloqy

The rheology of Parts A and B was evaluated on a strain controlled rheometer, TA Instrument ARES, under conditions as specified in Example 1. The rheology results are summarized in Tables F and G below.

TABLE F
Rheology of Part A

| Freq | Eta* | G′ | G″ | | Strain | Time |
|---|---|---|---|---|---|---|
| (rad/s) | (P) | (dyn/cm²) | (dyn/cm²) | tan _delta | (%) | (s) |
| 0.01 | 930,240 | 5,953 | 7,148 | 1.201 | 0.099 | 320 |
| 0.032 | 615,199 | 15,801 | 11,349 | 0.718 | 0.099 | 2127 |
| 0.1 | 299,864 | 26,267 | 14,464 | 0.551 | 0.099 | 2464 |
| 0.316 | 134,376 | 38,801 | 17,326 | 0.447 | 0.099 | 2570 |
| 1 | 55,746 | 52,008 | 20,070 | 0.386 | 0.099 | 2604 |
| 3.162 | 22,880 | 68,248 | 24,023 | 0.352 | 0.100 | 2617 |
| 10 | 9,142 | 86,343 | 30,042 | 0.348 | 0.100 | 2626 |
| 31.623 | 3,641 | 107,925 | 40,086 | 0.371 | 0.100 | 2634 |
| 100 | 1,493 | 137,701 | 57,608 | 0.418 | 0.076 | 2642 |

TABLE G
Rheology of Part B

| Freq | Eta* | G′ | G″ | | Strain | Time |
|---|---|---|---|---|---|---|
| (rad/s) | (P) | (dyn/cm²) | (dyn/cm²) | tan _delta | (%) | (s) |
| 0.01 | 3,695,721 | 28,707 | 23,276 | 0.811 | 0.099 | 320 |
| 0.032 | 2,073,683 | 57,403 | 31,704 | 0.552 | 0.099 | 2285 |
| 0.1 | 909,257 | 83,574 | 35,817 | 0.429 | 0.099 | 2736 |
| 0.316 | 374,890 | 111,776 | 39,501 | 0.353 | 0.099 | 2843 |
| 1 | 147,786 | 141,379 | 43,042 | 0.304 | 0.099 | 2877 |
| 3.162 | 56,474 | 171,988 | 48,104 | 0.280 | 0.099 | 2890 |
| 10 | 21,318 | 205,950 | 55,048 | 0.267 | 0.100 | 2899 |
| 31.623 | 8,113 | 248,205 | 64,928 | 0.262 | 0.098 | 2908 |
| 100 | 3,054 | 294,402 | 81,078 | 0.275 | 0.072 | 2915 |

To evaluate the rheology of the crosslinked material (Parts A and B combined), equal Parts A and Parts B were mixed using a dual asymmetric centrifuge mixer. The combined material was mixed for 16 seconds then mixed with a spatula. The material was then placed back in the dual asymmetric centrifuge mixer and mixed two more times for 16 seconds each (spatula mixed after second mix). The combined parts of A and B were cured on a strain controlled rheometer, TA Instrument ARES, at 130° C. for 20 minutes. After cooling, the rheology was evaluated across a frequency range of 0.01 rad/s to 100 rad/s (log scale—2 points per decade) at 3.0% strain and 30° C. (gap=1.4 mm). The rheology of cured Parts A and B is shown in Table H below.

TABLE H
Rheology of Cured Parts A and B

| Freq | Eta* | G′ | G″ | | Strain | Time |
|---|---|---|---|---|---|---|
| (rad/s) | (P) | (dyn/cm²) | (dyn/cm²) | tan _delta | (%) | (s) |
| 0.01 | 14,834,070 | 125,665 | 78,824 | 0.627 | 2.973 | 320 |
| 0.032 | 5,976,770 | 162,107 | 97,176 | 0.599 | 2.967 | 1430 |
| 0.1 | 2,484,214 | 212,120 | 129,299 | 0.610 | 2.959 | 1765 |
| 0.316 | 1,066,134 | 285,138 | 179,891 | 0.631 | 2.947 | 1871 |
| 1 | 470,648 | 393,471 | 258,244 | 0.656 | 2.928 | 1905 |
| 3.162 | 212,281 | 558,428 | 372,545 | 0.667 | 2.904 | 1920 |
| 10 | 96,374 | 802,731 | 533,314 | 0.664 | 2.870 | 1928 |
| 31.623 | 43,931 | 1,172,111 | 745,739 | 0.636 | 2.827 | 1937 |
| 100 | 19,824 | 1,714,280 | 995,588 | 0.581 | 2.721 | 1945 |

While the invention is susceptible to various modifications and alternative forms, specific embodiments have been shown by way of example in the drawings and described in detail herein. It should be understood, however, that the invention is not intended to be limited to the particular forms disclosed. Rather, the invention is to cover all modifications, equivalents, and alternatives falling within the spirit and scope of the invention as defined by the appended claims.

1. A pattern coating process for making a patterned silicone adhesive gel that can be pattern coated directly onto an absorbent substrate comprising
(1) mixing
a. at least one organopolysiloxane,
b. at least one SiH-containing organopolysiloxane, and
c. a thixotropic additive comprising a hydrogenated vegetable oil in the presence of a hydrosilyation catalyst to form a silicone composition, wherein the silicone composition exhibits:
i. viscosity ranging from about 7000 cP to about 5,000,000 cP, and
ii. shear thinning behavior, as determined by the rheological profile,
(2) pattern coating the silicone composition directly onto the absorbent substrate in a predetermined pattern;
(3) curing the silicone composition to form a patterned silicone adhesive gel which maintains the predetermined pattern, wherein the patterned silicone adhesive gel exhibits:
i. adhesiveness ranging from about 0.2 N to about 4 N, and
ii. cohesive strength, as determined by the peel adhesion test thereby allowing the pattern of the pattern coating to be maintained upon application.

2. The pattern coating process of claim 1, wherein the thixotropic additive is a trihydroxystearin.

3. The pattern coating process of claim 1, wherein the thixotropic additive has the formula: 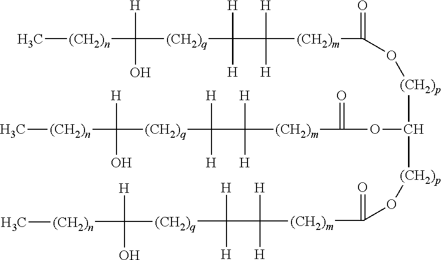
wherein m, n, p, and q are each, independently, an integer ranging from 1-10.

4. The pattern coating process of claim 3, wherein p and q are each, independently, an integer ranging from 1-3 and m and n are each, independently, an integer ranging from 4-10.

5. The pattern coating process of claim 1, wherein the thixotropic additive is present in amounts ranging from about 1 wt. % to about 15 wt. % based on the total wt. % of the silicone composition.

6. The pattern coating process of claim 1, further comprising mixing (d) a silicon-based resin with the at least one organopolysiloxane, the at least one SiH-containing organopolysiloxane, and the thixotropic additive.

7. A medical dressing comprising an absorbable substrate pattern coated with the silicone composition prepared by the pattern coating process of claim 1.

8. The medical dressing of claim 7, wherein the predetermined pattern is discontinuous.

9-10. (canceled)

11. A silicone composition having high-density particles suspended in an adhesive gel, the silicone composition comprising:
a. at least one organopolysiloxane,
b. at least one SiH-containing organopolysiloxane, and
c. about 0.1 to about 3 wt. % of a hydrogenated vegetable oil,
whereby the silicone composition is capable of suspending the high-density particles in the adhesive gel.

12. The silicone composition of claim 11, wherein the hydrogenated vegetable oil has the formula: 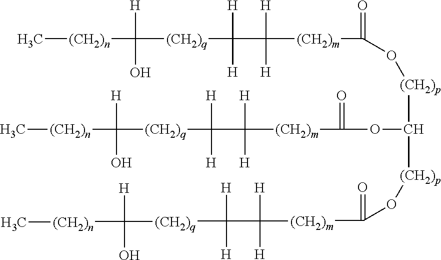
wherein m, n, p, and q are each, independently, an integer ranging from 1-10.

13. The silicone composition of claim 11, wherein the high-density particles are metal particles.

14-17. (canceled)

18. The pattern coating process of claim 1, wherein the predetermined pattern is discontinuous.

19. A method of preparing a medical dressing containing a patterned silicone adhesive gel that can be pattern coated directly onto an absorbent substrate comprising:
(1) mixing (a) at least one organopolysiloxane, (b) at least one SiH-containing organopolysiloxane, and (c) a thixotropic additive comprising a hydrogenated vegetable oil in the presence of a hydrosilyation catalyst to form a silicone composition, wherein the silicone composition exhibits:
i. viscosity ranging from about 7000 cP to about 5,000,000 cP, and
ii. shear thinning behavior, as determined by the rheological profile;
(2) pattern coating the silicone composition directly onto the absorbent substrate of the medical dressing in a predetermined pattern; and
(3) curing the silicone composition to form a patterned silicone adhesive gel which maintains the predetermined pattern, wherein the patterned silicone adhesive gel exhibits:
i. adhesiveness ranging from about 0.2 N to about 4 N, and
ii. cohesive strength, as determined by the peel adhesion test thereby allowing the pattern of the pattern coating to be maintained upon application.

20. The method of claim 19, wherein the thixotropic additive is a trihydroxystearin.

21. The method of claim 19, wherein the thixotropic additive has the formula: 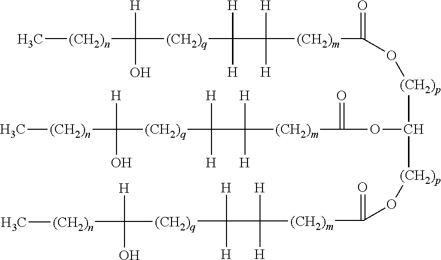
wherein m, n, p, and q are each, independently, an integer ranging from 1-10.

22. The method of claim 19, wherein the thixotropic additive is present in amounts ranging from about 1 wt. % to about 15 wt. % based on the total wt. % of the silicone composition.

23. The method of claim 19, further comprising mixing (d) a silicon-based resin with the at least one organopolysiloxane, the at least one SiH-containing organopolysiloxane, and the thixotropic additive.

24. The method of claim 19, wherein the predetermined pattern is discontinuous.

25. The method of claim 19, further comprising mixing one or more hydrophilic additives, fillers, pigments, actives or pharmaceuticals with the at least one organopolysiloxane, the at least one SiH-containing organopolysiloxane, and the thixotropic additive.

26. A medical dressing comprising a patterned silicone adhesive gel prepared by the method of claim 19.
